(19) United States
(12) Patent Application Publication
(10) Pub. No.: US 2018/0228827 A1
(43) Pub. Date: Aug. 16, 2018

(54) FIXED-DOSE COMBINATIONS OF ANTIVIRAL COMPOUNDS

(71) Applicant: MERCK SHARP & DOHME CORP., RAHWAY, NJ (US)

(72) Inventors: William A Marinaro, Scotch Plains, NJ (US); David Harris, New Providence, NJ (US); James DiNunzio, Bridgewater, NJ (US); Sutthilug Sotthivirat, Lansdale, PA (US); Sundeep Sudish Dhareshwar, Whippany, NJ (US); Gerard Ross Klinzing, Wilmington, DE (US); Jesse Lee Kuiper, North Wales, PA (US); Yung-Chi Lee, New Providence, NJ (US); Craig Alfred McKelvey, Ambler, PA (US); Michael McNevin, Basking Ridge, NJ (US); Li Xiong, Cedar Knolls, NJ (US)

(73) Assignee: MERCK SHARP & DOHME CORP., RAHWAY, NJ (US)

(21) Appl. No.: 15/750,058

(22) PCT Filed: Jul. 29, 2016
(86) PCT No.: PCT/US2016/044608

§ 371 Date: Feb. 2, 2018

Related U.S. Application Data
(60) Provisional application No. 62/200,955, filed on Aug. 4, 2015.

(51) Int. Cl. A61K 31/7072; A61K 31/427; A61K 9/20; A61K 9/28; A61K 9/14

(52) U.S. Cl. CPC A61K 31/7072; A61K 31/427; A61K 9/2095; A61K 9/145; A61K 9/2013; A61K 9/28; A61K 9/2054

(57) ABSTRACT

The present disclosure is directed to compositions comprising blended materials comprising a substantially crystalline HCV nucleotide polymerase inhibitor; a solid dispersion formulation, which comprises an HCV NS5a inhibitor or a pharmaceutically acceptable salt thereof, one or more pharmaceutically acceptable polymers or a mixture thereof, and optionally one or more pharmaceutically acceptable surfactants or a mixture thereof; and optionally one or more pharmaceutically acceptable surfactants or a mixture thereof; and optionally one or more excipients. The present disclosure is also directed to oral dosage forms, such as tablets or capsules comprising the disclosed blended compositions comprising the disclosed solid dispersion formulations, and the methods for making these solid dispersion formulations, and pharmaceutical compositions

### CROSS-REFERENCE TO RELATED APPLICATIONS

Not Applicable

### FIELD OF THE INVENTION

The instant invention relates to pharmaceutical formulations that are useful for the treatment of diseases and disorders caused by hepatitis C virus (“HCV”). In particular, the pharmaceutical formulations are fixed-dose combinations that comprise solid dispersion formulations of two or more antiviral compounds.

### BACKGROUND OF THE INVENTION

HCV infection is a major health problem that leads to chronic liver disease, such as cirrhosis, and hepatocellular carcinoma, in a substantial number of infected individuals. Current treatments for HCV infection include immunotherapy with recombinant interferon-α alone or in combination with the nucleoside analog ribavirin. Potential treatments for HCV infection have been discussed in the different references including Balsano, 8(4) MINI REV. MED. CHEM. 307-318, 2008; Rönn et al., 8 CURRENT TOPICS IN MEDICINAL CHEMISTRY 533-562, 2008; Sheldon et al., 16(8) EXPERT OPIN. INVESTIG. DRUGS 1171-1181, 2007; and De Francesco et al., 58 ANTIVIRAL RESEARCH 1-16, 2003. Several virally encoded enzymes are putative targets for therapeutic intervention, including a metalloprotease (non-structural (NS) 2-3), a serine protease (NS3, amino acid residues 1-180), a helicase (NS3, full length), an NS3 protease cofactor (NS4A), a membrane protein (NS4B), a zinc metalloprotein (NS5A), and an RNA-dependent RNA polymerase (NS5B).

One potential avenue for treatment is combination therapy, in which two or more antiviral agents are co-administered, with each antiviral agent acting on one or more of these non-structural regions as therapeutic targets. The combination of two or more antiviral agents acting on different non-structural regions may provide a combination drug product having additive effects for viral load suppression. Indeed, the landscape for treatment of HCV is trending towards an all-oral, direct-acting antiviral regimen that is active against all HCV genotypes, and combination drug products that comprise two or more HCV antiviral agents, each acting on a different therapeutic target, may form a crucial component of an all-oral regimen.

The HCV NS5B enzyme is an RNA-dependent RNA polymerase that has long been considered a prime drug target because it is essential for viral replication. One class of HCV polymerase inhibitor compounds include nucleoside analog compounds such as (2R)-isopropyl 2-(((((2R,3R,4R,5R)-4-chloro-5-(2,4-dioxo-3,4-dihydropyrimidin-1(2H)-yl)-3-hydroxy-4-methyltetrahydrofuran-2-yl)methoxy)(phenoxy)phosphoryl)amino)propanoate, which is shown below as Compound I:

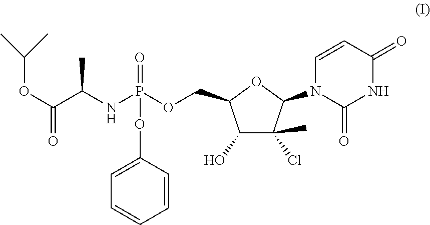 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]

Compound I is described in PCT International Patent Application Publication Nos. WO2013/177219 and WO2014/058801. Compound I is a selective HCV polymerase inhibitor.

Another identified target for therapeutic intervention is the HCV NS5A non-structural protein, which is described, for example, in Seng-Lai Tan & Michael G. Katze, 284 VIROLOGY 1-12 (2001); and in Kyu-Jin Park et al., 278(33) J. BIO. CHEM. 30711 (2003). A non-structural protein, NS5A is an essential component for viral replication, and assembly. Mutations in NS5A at or near known sites of phosphorylation can affect the ability for high-level replication in cell-culture systems, suggesting an important role for NS5A phosphorylation in viral replication efficiency. Inhibitors of the phosphorylation of NS5A can lead to reduced viral RNA replication.

NS5A inhibitor compounds include compounds such as dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, which is shown below as Compound II:

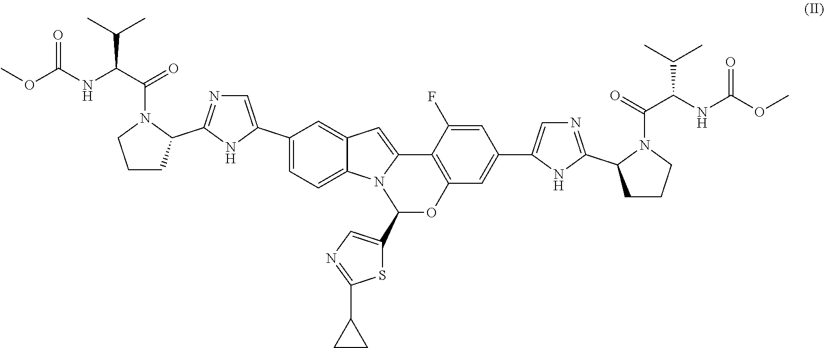

Compound II is described in PCT International Patent Application Publication No. WO2014/110705.

Compound II, a weak base, has two basic sites, which protonate at low pH giving rise to a sharp pH-dependent solubility profile, particularly between pH 1-4. A normal human stomach has a pH in a range from 1-3, usually closer to 2, although it varies depending on the type, and quantity of food ingested. The steep pH-dependent solubility profile has practical implications for dissolution, and absorption of Compound II, as for the dissolution, and absorption of weak bases in general, in the gastrointestinal tract of patients. Specifically, the amount of drug dissolved from formulations of weakly basic compounds can vary as the gastric pH fluctuates within this normal range, which in turn can lead to more variable and potentially lower absorption. See E. Lahner et al., 29 ALIMENTARY PHARMACOL. THER. 1219-1229 (2009); T. L. Russell et al., 11(1) PHARM. RES. 136-143 (1994); G. Krishna et al., 53(3) ANTIMICROB. AGENTS CHEMOTHER. 958-966 (2009).

Patients may exhibit a significantly higher gastric pH, known as achlorhydria, which can arise due to age or concomitant disease, for example, or which can be the result of other drug treatments (e.g., proton pump inhibitors, H2 receptor antagonists). See A. Mitra & F. Kesisoglou, 10 MOL. PHARM. 3970-3979 (2013). Absorption of weakly basic drugs that have low solubility at higher pH (e.g., ketoconazole, itraconazole, atazanavir, cefpodoxime, enoxacin, dipyridamole, nifedipine, and digoxin) has been shown to be impaired due to this condition. See E. Lahner et al., 29 ALIMENTARY PHARMACOL. THER. 1219-1229 (2009).

Because of the importance of gastric pH in driving dissolution, absorption, and ultimately efficacy of Compound II, it is imperative to develop formulations that can minimize or mitigate the effects of increased gastric pH on Compound II's bioavailability. Such formulations may prove particularly useful in the treatment of HIV patients who are coinfected with HCV. About one-quarter of HIV-infected persons in the United States are also infected with HCV, and these patients tend to have higher gastric pH. See HIV and Viral Hepatitis Fact Sheet, Centers for Disease Control and Prevention (March 2014), available online at http://www.edc.gov/hepatitis/Populations/PDFs/HIVandHep-FactSheet.pdf. Similarly, these formulations would be useful in the treatment of HCV in patients who are also being treated with drugs that modulate gastric pH (e.g., proton pump inhibitors).

Solid dispersion formulations have been used previously to promote the oral absorption of poorly water soluble active pharmaceutical ingredients (APIs) (see Ford, 61 PHARM. ACTA HELV. 69-81 (1986)), and to minimize the effect of achlorhydria for weak bases (see M. A. Alam et al., 9(11) EXPERT OPIN. DRUG DELIVERY 1419-1440 (2012); A. Mitra et al., 8 MOL. PHARM. 2216-2223 (2011)). Solid dispersion formulations are compositions in which APIs are dispersed into biologically-inert matrices. Solid solutions, defined as solid dispersions in which the API forms a homogeneous or nearly homogeneous glass when dispersed into the excipient matrix at the molecular level, are of particular interest in the oral delivery of compounds that are poorly water soluble and/or sensitive to gastric pH. The broader category of solid dispersions also includes systems in which the API is dispersed as microfine crystalline or amorphous domains within the carrier. It should be noted that in many scientific and technical publications, the terms “solid solution” and “solid dispersion” have been used largely interchangeably; this reflects, among other things, the difficulty in ascertaining the level of the dispersion at the sub-microscopic level. Solid dispersion formulations as described above may provide increased absorption of APIs and/or enhanced insensitivity to variations in gastric pH relative to crystalline forms of the API. There remains a need for formulations that provide increased absorption and/or enhanced insensitivity to variations in gastric pH relative to other formulations containing amorphous forms of the API.

The use of solid solution or solid dispersion formulations to effectively promote oral drug absorption continues to grow, but their design remains largely unpredictable. There remains a need for solid dispersion formulations of drug substances that may provide effective absorption following oral administration, which is useful to reduce pill burden (e.g., the number of tablets administered), regimen complexity (e.g., eliminating the need to administer with food or without food), and facilitate co-dosing with other medications, such as antacid medications. Formulations with this type of enhanced absorption will ultimately improve compliance and, therefore, efficacy.

Combining one or more API, which may be in the form of a solid dispersion formulation, into a single dosage form may couple the advantages provided by the individual solid dispersions, while providing the additive effect of dosing two or more drug substances. However, designing an effective combination of solid dispersion formulations is dependent on the impact on dosage form characteristics of the properties of the individual solid dispersion formulations.

The current invention relates to novel formulations based on the combination of Compound I with a solid dispersion formulation of Compound II, which may provide improved oral absorption, confer insensitivity to higher gastric pH, enhance dissolution rate, and/or maintain higher supersaturation of Compound II relative to their individual crystalline or amorphous forms.

### SUMMARY OF THE INVENTION

The present disclosure relates to blended compositions comprising (a) (2R)-isopropyl 2-(((((2R,3R,4R,5R)-4-chloro-5-(2,4-dioxo-3,4-dihydropyrimidin-1(2H)-yl)-3-hydroxy-4-methyltetrahydrofuran-2-yl)methoxy)(phenoxy)phosphoryl)amino)propanoate (Compound I):

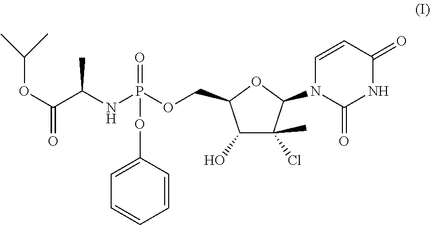 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]

or a pharmaceutically acceptable salt thereof; (b) a solid dispersion formulation, which comprises (i) dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate (Compound II):

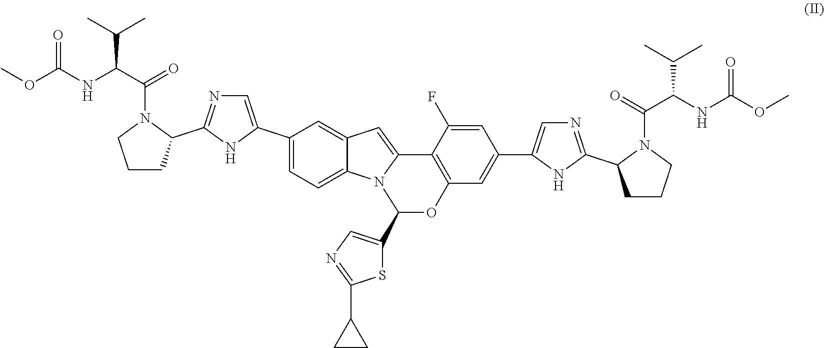

or a pharmaceutically acceptable salt thereof; (ii) one or more pharmaceutically acceptable polymers or a mixture thereof; and (iii) optionally one or more pharmaceutically acceptable surfactants or a mixture thereof; and wherein Compound II, and the one or more surfactants, if present, are dispersed in a polymer matrix formed by the one or more pharmaceutically acceptable polymers; and (c) optionally one or more of a diluent, disintegrant, salt, lubricant, and glidant. In embodiments, compositions of the disclosure may provide improved oral bioavailability, and/or insensitivity to gastric pH.

Other embodiments, aspects, and features of the present invention are either further described in or will be apparent from the ensuing description, examples, and appended claims.

### BRIEF DESCRIPTION OF THE DRAWINGS

FIG. 1 provides a schematic representation of the process for preparing wet-granulated Conventional Formulation 2 of Compound II, as set forth in Example 2.

FIG. 2 provides a schematic representation of the formulation process for preparing Solid Dispersion Formulation 3 of Compound II, as set forth in Example 3.

FIG. 3 provides a schematic representation of the process for preparing Tablet Formulation 1 of Compound II, as set forth in Example 3.

FIG. 4 provides a schematic representation of the formulation process for preparing the Solid Dispersion Formulation 4 of Compound II, as set forth in Example 4.

FIG. 5 provides a schematic representation of the process for preparing Tablet Formulation 2 of Compound II, as set forth in Example 4.

FIG. 6 provides a schematic representation of the formulation process for preparing fixed-dose combination Tablet Formulation 3, as set forth in Example 5.

FIG. 7 provides a schematic representation of the formulation process for preparing fixed-dose combination Tablet Formulation 4, as set forth in Example 6.

### DETAILED DESCRIPTION OF THE INVENTION

The instant disclosure is directed to blended compositions comprising one substantially crystalline API, and two solid dispersion formulations each comprising a different API, and optionally one or more excipients. The disclosure is also directed to oral dosage forms, such as tablets or capsules comprising such blended compositions that comprise such solid dispersion formulations.

Compound I is an HCV NS5B polymerase inhibitor, specifically a nucleoside analog, which is administered as a phosphoramidate prodrug that is converted in vivo to the corresponding nucleoside triphosphate, which is the active form. The compound is formulated in a predominantly crystalline form, and should be capable of forming salts, although no salts have been prepared to date. It is unlikely that the compound would be formulated as a salt, however, since the conditions used to form salts would likely cause some degradation of the compound. Compound I is formulated in a substantially crystalline form.

Compound II is a weak base, with two basic sites, which protonate at low pH giving rise to pH dependent solubility profile. This pH-dependent solubility could significantly impair the amount of drug dissolved from formulations of weakly basic compounds in patients with elevated gastric pH, which in turn could lead to potentially lower absorption. See E. Lahner et al., 29 ALIMENTARY PHARMACOL. THER. 1219-1229 (2009); T. L. Russell et al., 11(1) PHARM. RES. 136-143 (1994); G. Krishna et al., 53(3) ANTIMICROB. AGENTS CHEMOTHER. 958-966 (2009). In order to mitigate variability in absorption of Compound II due to elevated gastric pH, solid dispersion formulations of Compound II may be formulated at a drug loading up to approximately 20% in combination with pharmaceutically suitable polymers and surfactants.

The use of solid dispersion formulations, and, particularly, solid solutions, to promote the oral absorption of poorly water-soluble APIs is known. See, e.g, Ford, 61 PHARM. ACTA. HELV. 69-88 (1986); Craig, 231 INT. J. PHARM. 131-144 (2002). As discussed above, it is believed that these solid solutions may improve the absorption of orally administered APIs by enhancing the dissolution of the API, causing transient supersaturation of the API with respect to a lower energy phase (e.g., crystalline API), or both. In general, solid solutions are believed to enable drug absorption by enhancing the dissolution rate, and/or its extent.

Oral dosage forms, combining Compound I and the solid dispersion formulation of Compound II, may exhibit similar stability, and bioavailability for each of Compound I and Compound II as single-entity formulations.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range, and any value in the continuum there between. Numerical values provided herein, and the use of the term “about”, may include variations of ±1%, ±2%, ±3%, ±4%, ±5%, ±10%, ±15%, and ±20%, and their numerical equivalents.

As used herein, the term “one or more” item includes a single item selected from the list as well as mixtures of two or more items selected from the list.

As used herein, the term “amorphous” indicates that the material lacks a high degree of order on a molecular level and may exhibit the physical properties of a solid or a liquid, depending on the temperature of the material. Amorphous materials do not give X-ray diffraction patterns with distinctive sharp peaks.

As used herein, the term “crystalline” indicates that the material has a regular ordered internal structure at the molecular level when in the solid phase, and the crystalline material gives a distinctive X-ray diffraction pattern with defined peaks.

As used herein, the term “substantially amorphous” refers to a composition in which greater than 70%; or greater than 75%; or greater than 80%; or greater than 85%; or greater than 90%; or greater than 95%, or greater than 99% of the compound is amorphous. “Substantially amorphous” can also refer to material that has no more than about 20% crystallinity, or no more than about 10% crystallinity, or no more than about 5% crystallinity, or no more than about 2% crystallinity.

As used herein, the term “substantially crystalline” refers to a composition in which greater than 70%; or greater than 75%; or greater than 80%; or greater than 85%; or greater than 90%; or greater than 95%, or greater than 99% of the compound is crystalline. “Substantially crystalline” can also refer to material that has no more than about 20% crystallinity, or no more than about 10% amorphous, or no more than about 5% amorphous, or no more than about 2% amorphous.

The term “effective amount” indicates a sufficient amount to exert a therapeutic or prophylactic effect. For a patient who is infected with HCV, an effective amount is sufficient to achieve one or more of the following effects: reduce the ability of HCV to replicate, reduce HCV load, and increase viral clearance. For a patient who is not infected with HCV, an effective amount is sufficient to achieve one or more of the following: a reduced susceptibility to HCV infection and a reduced ability of the infecting virus to establish persistent infection for chronic disease.

The term “subject” (alternatively referred to herein as “patient”) as used herein refers to an animal, preferably a mammal, most preferably a human, who has been the object of treatment, observation, or experiment.

Each of Compound I and Compound II, as provided in the solid dispersion formulations of Compound II, the blended compositions, and/or the oral dosage forms described herein, independently may take the form of pharmaceutically acceptable salts. The term “pharmaceutically acceptable salt” refers to a salt of the parent compound that has activity and that is not biologically or otherwise undesirable (e.g., is neither toxic nor otherwise deleterious to the recipient thereof); also included in this term are complexes that comprise solvent molecules, and a salt of the parent compound. Suitable salts include acid addition salts that may, for example, be formed by mixing a solution of a compound with a solution of a pharmaceutically acceptable acid such as hydrochloric acid, sulfuric acid, acetic acid, trifluoroacetic acid, benzoic acid, phosphoric acid, methanesulfonic acid, naphthalene-1,5-disulfonic acid, and toluenesulfonic acid. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands, such as quaternary ammonium salts. Also, in the case of an acid (—COOH) or alcohol group being present, pharmaceutically acceptable esters can be employed to modify the solubility or hydrolysis characteristics of the compound.

The term “polymer” as used herein refers to a chemical compound or mixture of compounds consisting of repeating structural units created through a process of polymerization. Suitable polymers useful in this invention are described throughout. When specific polymers that are suitable for use in the compositions of the present invention are blended, the blends of such polymers may also be suitable. Thus, the term “polymer” is intended to include blends of polymers in addition to a single species of polymer.

In the embodiments described herein, any variable or component is as defined in the first instance where the variable or component occurs, unless otherwise indicated. When any variable or component occurs more than one time, its selection on each occurrence is independent of its selection at every other occurrence, unless it is expressly stated otherwise. Also, combinations of embodiments, variables or components are permissible only if such combinations result in stable formulations, blends, or oral dosage forms.

### Compound I

Compound I is provided in a form that is substantially crystalline. It may be formulated as a solid dosage form by blending or granulating with excipients, and compressed into tablets or filled into hard capsule shells. The granulation process may be a dry granulation process such as roller-compaction, or it may be a wet granulation process such as high-shear wet granulation or fluidized-bed granulation.

Examples of excipients that can be used in formulations of Compound I include, but are not limited to, binders, fillers, disintegrants, and lubricants. Binders suitable for use in the pharmaceutical compositions provided herein include, but are not limited to, starches, cellulose, and its derivatives (e.g., ethylcellulose, cellulose acetate, carboxymethyl cellulose calcium, sodium carboxymethyl cellulose, methylcellulose, hydroxypropyl methylcellulose), polyvinyl pyrrolidone, and mixtures thereof. Examples of fillers suitable for use in the pharmaceutical compositions provided herein include, but are not limited to, microcrystalline cellulose, powdered cellulose, mannitol, lactose, calcium phosphate, starch, pre gelatinized starch, and mixtures thereof. The binder or filler in pharmaceutical compositions is typically present in from about 50 to about 99 weight percent of the pharmaceutical composition or dosage form.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in the pharmaceutical compositions provided herein include, but are not limited to, croscarmellose sodium, crospovidone, sodium starch glycolate, potato or tapioca starch, pre gelatinized starch, other starches, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Lubricants are typically used in an amount of less than about 1 weight percent of the pharmaceutical compositions or dosage forms into which they are incorporated.

Compressed tablet formulations of Compound I may optionally be film-coated to provide color, light protection, and/or taste-masking. Tablets may also be coated so as to modulate the onset, and/or rate of release in the gastrointestinal tract, so as to optimize or maximize the biological exposure of the patient to the API. Hard capsule formulations of Compound I may be produced by filling a blend or granulation of Compound I into shells consisting of, for example, gelatin, or hypromellose.

### Solid Dispersion Formulation of Compound II

A solid dispersion formulation comprises (a) Compound II or a pharmaceutically acceptable salt thereof; (b) one or more pharmaceutically acceptable polymers; and (c) optionally one or more pharmaceutically acceptable surfactants.

Compound II, or a pharmaceutically acceptable salt thereof, is present in a concentration of from about 5% w/w to about 50% w/w. In particular instances, Compound II, or a pharmaceutically acceptable salt thereof, is present in a concentration of from about 10% w/w to about 40% w/w, or about 20% w/w. All other variables are as provided above.

Compound II may be in the form of a pharmaceutically acceptable salt. In instances, the pharmaceutically acceptable salt of Compound II may be a bis-tosylate salt of Compound II. In additional instances, Compound II may also be anhydrous or in the form of a hydrate or solvate.

The one or more pharmaceutically acceptable polymers may enhance the absorption of the API when used in the solid dispersion formulations described herein. The one or more pharmaceutically acceptable polymers are selected from the group consisting of cellulosic polymers, and vinyl pyrrolidone/vinyl acetate copolymers.

Cellulosic polymers include cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate, and hydroxypropylmethylcellulose phthalate, hydroxypropylmethylcellulose succinate, or hydroxypropylmethylcellulose acetate succinate (HPMCAS)). Commercially available examples of these include hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P 55.

The pharmaceutically acceptable polymer may be vinyl pyrrolidone/vinyl acetate copolymers. In particular instances, the pharmaceutically acceptable polymer is copovidone, a copolymer of 1-vinyl-2-pyrrolidone and vinyl acetate in the mass proportion of 3:2. Other useful copolymers contain vinyl pyrrolidone and vinyl acetate in ratios of, for example, 90:10, 80:20, 70:30, and 50:50. The amount of vinyl pyrrolidone can range from about 40% up to about 99.9%, and the amount of vinyl acetate can range from about 0.1% up to about 60%. Other vinyl polymers, and copolymers having substituents that are hydroxy, alkyl, acyloxy, or cyclic amides include polyethylene polyvinyl alcohol copolymers; and polyvinyl caprolactam-polyvinyl acetate-polyethylene glycol graft copolymer (SOLUPLUS®, BASF Corp.). Commercially available copolymers of vinyl pyrrolidone and vinyl acetate include PLASDONE® S630 (Ashland, Inc., Covonton, Ky.), and KOLLIDON® VA 64 (BASF Corp., Florham Park, N.J.), which contain vinyl pyrrolidone and vinyl acetate in a 60:40 ratio. Other copolymers of vinyl pyrrolidone and vinyl acetate can also be used in the invention. Preferably, the copolymer contains at least 40% vinyl pyrrolidone, although smaller amounts of vinyl pyrrolidone can also be utilized.

The one or more pharmaceutically acceptable polymer may be non-ionic.

The one or more pharmaceutically acceptable polymers are selected from the group consisting of cellulosic polymers and vinyl pyrrolidinone/vinyl acetate copolymers. In particular aspects of this embodiment, the one or more pharmaceutically acceptable polymer is selected from the group consisting of hydroxypropylmethyl cellulose (HPMC), hydroxypropylmethyl cellulose acetate succinate (HPMCAS), and hydroyxpropylmethyl cellulose phthalate (HPMCP). In particular instances, the one or more pharmaceutically acceptable polymer is HPMC. All other variables are as provided above.

The one or more pharmaceutically acceptable polymers are present in a concentration of from about 50% w/w to about 95% w/w. In instances, the one or more pharmaceutically acceptable polymers are present in a concentration of from about 50% w/w to about 90% w/w, or about 70% w/w. All other variables are as provided above.

The action of polymers may be improved by the presence of one or more pharmaceutically acceptable surfactants. The surfactants can increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Surfactants may also facilitate absorption of APIs by altering API permeability, and/or efflux directly. See, e.g., Yu et al., 16 PHARM RES. 1812-1817 (1999). Non-limiting examples of pharmaceutically acceptable surfactants that are suitable for the present invention include polyoxyethylene castor oil derivates, e.g. polyoxyethyleneglycerol triricinoleate or polyoxyl 35 castor oil (CREMOPHOR® EL; BASF Corp.) or polyoxyethyleneglycerol oxystearate such as polyethylenglycol 40 hydrogenated castor oil (CREMOPHOR® RH 40, also known as polyoxyl 40 hydrogenated castor oil or macrogolglycerol hydroxystearate) or polyethylenglycol 60 hydrogenated castor oil (CREMOPHOR® RH 60); or polysorbates or mono fatty acid esters of polyoxyethylene sorbitan, such as a mono fatty acid ester of polyoxyethylene (20) sorbitan, e.g. polyoxyethylene (20) sorbitan monooleate (commercially available as TWEEN® 80), polyoxyethylene (20) sorbitan monostearate (commercially available as TWEEN® 60), polyoxyethylene (20) sorbitan monopalmitate (commercially available as TWEEN® 40), or polyoxyethylene (20) sorbitan monolaurate (commercially available as TWEEN® 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate; sorbitan fatty acid mono esters such as sorbitan mono laurate (commercially available as SPAN® 20), sorbitan monooleate, sorbitan monopalnitate (commercially available as SPAN® 40), or sorbitan stearate; D-alpha-tocopheryl polyethylene glycol 1000 succinate (TPGS); or a combination or mixture thereof. Other non-limiting examples of suitable surfactants include anionic surfactants, e.g. docusate potassium, docusate sodium, docusate calcium, and sodium lauryl sulfate (SLS). Other suitable surfactants include, but are not limited to, block copolymers of ethylene oxide and propylene oxide, also known as polyoxyethylene polyoxypropylene block copolymers or polyoxyethylene polypropyleneglycol, such as POLOXAMER® 124, POLOXAMER® 188, POLOXAMER® 237, POLOXAMER® 388, or POLOXAMER® 407 (BASF Corp.). As described above, a mixture of surfactants can be used in a solid composition of the present invention. In particular instances, the surfactant is selected from the group consisting of sodium lauryl sulfate (SLS), D-α-tocopheryl polyethylene glycol 1000 succinate (TPGS), or nonionic ethoxylated alcohols like polysorbate or poloxamer. In aspects of this solid dispersion formulation, the surfactant may be selected from the group consisting of sodium lauryl sulfate (SLS), D-α-tocopheryl polyethylene glycol 1000 succinate (TPGS), or nonionic ethoxylated alcohols like polysorbate or poloxamer. In particular instances, the one or more pharmaceutically acceptable surfactant is TPGS. All other variables are as provided above.

The one or more pharmaceutically acceptable surfactant may be present in a concentration of from about 2% w/w to about 20% w/w. In particular instances, the one or more pharmaceutically acceptable surfactant is present in a concentration of from about 5% w/w to about 15% w/w, or about 10% w/w. All other variables are as provided above.

The solid dispersion formulation may be in the form of particles, with all other variables as provided above.

The solid dispersion formulation described herein relates to solid dispersion formulations produced by solvent removal (e.g., spray drying), introduction of an antisolvent (e.g., precipitation), addition of heat together with mixing (e.g., extrusion), mechanical activation or other means (e.g., to produce a “solid dispersion intermediate”). That is, the solid dispersion formulation may be formed by a process selected from spray drying, and extrusion, such as hot melt extrusion, of the composition. In particular instances, the solid dispersion formulation comprises particles of the composition formed by spray drying.

The solid dispersion formulation described herein may be prepared by processes that are suitable for causing Compound II to form an amorphous dispersion in the polymer such that the drug is generally amorphous or dissolved in the polymer or a component of the composition, such as a surfactant. The dispersions are stable, and the drug does not form crystals or other insoluble particles. Such methods include solution methods, such as spray drying, spray coating, freeze drying, and evaporation of a co-solvent under vacuum or by heating a solution of polymer and drug. Such methods also include methods that blend the solid drug with the polymer in the molten state, such as hot melt extrusion, and methods of compounding the solid non-molten polymer and drug under heat and pressure to form a dispersion. If the dispersion is effectively a homogeneous molecular dispersion of the individual components, it may also be described as a solid solution, a specific subclass of solid dispersions.

Spray drying is well known (see, e.g., Masters, Spray Drying Handbook, 1991, 5^{th} edition, Longman Scientific & Technical), and widely practiced in a variety of industrial applications including spray drying of milk (see, e.g., U.S. Pat. No. 4,187,617), and pharmaceutical products (see, e.g., U.S. Pat. No. 6,763,607). To produce solid dispersion compositions by spray drying, the polymer, drug, and optional surfactant, are dissolved in a solvent, and then are sprayed through a nozzle as a fine spray into a chamber, where the solvent is evaporated quickly to make particles comprising polymer, drug, and surfactant. Ideally, the solvent is any solvent in which all of the components of the composition are soluble, and that is readily evaporated in a spray dryer. The solvent should also be suitable for use in preparing pharmaceutical compositions. In certain embodiments of the invention, the use of mixed-solvent systems, particularly those containing a combination of water, and another solvent, are necessary to facilitate the production of solid dispersion intermediates containing Compound II, an absorption enhancing polymer or polymer(s), and optionally a surfactant.

Useful solvents for spray drying include water, acetone, ethanol, methanol, dichloromethane, isopropanol, and tetrahydrofuran (THF). In aspects, the mixed-solvent system consists of a first solvent, and a second solvent, in which the first solvent may be selected from the group consisting of acetone, ethanol, methanol, dichloromethane, isopropanol, and tetrahydrofuran (THF); the second solvent is water. In particular aspects, the first solvent may be selected from the group consisting of ethanol, methanol, and acetone; the second solvent is water. In specific instances, the first solvent is acetone, and the second solvent is water. The proportions of the first solvent to second solvent may be about 90:10, about 80:20, about 70:30, or about 60:40. Mixed-solvent systems are described in PCT International Patent Application Publication No. WO2007/109605 and U.S. Patent Application Publication No. US2007/0026083. Solids loading, which usually refers to the concentration of solid components in the spray drying solvent system, does not typically exceed 50%, and depends on solution properties, such as solubility, stability, and viscosity. The solids, comprising Compound II, the pharmaceutically acceptable polymer, and surfactant, are present in the spray drying solution in a concentration of from about 5% w/w to about 25% w/w, based on the solubility, stability, and viscosity of the solution. In particular instances, the solids are present in the solution in a concentration of from about 10% w/w to about 20% w/w.

Following formation of a solid dispersion formulation, the resulting spray dried intermediate can undergo a secondary drying step to remove residual solvents. This secondary drying unit operation can occur in a static dryer or agitated dryer. Gas, humidified gas, or vacuum may be applied to the material in the secondary dryer, and such application can be useful in more rapidly removing residual solvents that remain in the spray-dried intermediate. See, e.g., European Patent Application No. EP1855652 A2 (and references therein), and PCT International Patent Application Publication No. WO2008/012617A1 (and references therein).

In hot melt extrusion, the polymer, drug, and optional surfactant may be either premixed together (e.g., via a wet or dry granulation process) or fed as independent feed streams into the extruder (see Polymer Extrusion 4^{th} Edition by Chris Rauwendaal 2001, Hanser Gardner Publications, Inc., Cincinnati, Ohio or Schenck et al., (2010), Achieving a Hot Melt Extrusion Design Space for the Production of Solid Solutions, in Chemical Engineering in the Pharmaceutical Industry: R&D to Manufacturing (ed. D. J. am Ende), John Wiley & Sons, Inc., Hoboken, N.J., USA). In accordance with this embodiment, any means for preparing a melt in any convenient apparatus in which an admixture of Compound II, a polymer, and optionally a surfactant can be heated, and optionally mixed can be used. Solidification can be carried out by cooling the melt. Once a solid is obtained, the solid can be further mechanically processed to provide a convenient form for incorporation into a medicament, for example, tablets or capsules.

It will be appreciated that other methods of preparing a melt, solidifying it, and forming the solid into conveniently sized particles can be utilized without departing from the spirit of the invention. For example, compositions of the invention may be prepared using an extruder. When an extruder is employed to prepare compositions of the invention, the material may be introduced into the extruder either in a pre-flux state, that is, as a dry admixture, or in a fluxed state, that is in a melted, plastic, or semi-solid state achieved after the application of sufficient heat to the admixture to cause Compound II to dissolve in the polymer, optionally when a fluxed charge is prepared, blending may be employed during heating to promote uniformity of the fluxed material.

If the material is introduced to the extruder in a fluxed state, residence time in the extruder is selected to be just sufficient to ensure homogeneity of the composition, and the temperature is preferably maintained in the extruder at a level just sufficient to insure that the material maintains its plasticity so that it can be extruded into a conveniently shaped extrudate. If the material is introduced into an extruder in a pre-flux state, the extruder components, for example, the barrels, and any mixing chamber present in the equipment, will be maintained at a temperature sufficient to promote fluxing of the admixture. Temperatures selected for use in processing a composition will also take into account that blending occurring within the extruder equipment, for example, in a mixing section of the barrels, will also contribute to localized fluxing of the admixture by imparting shear-stresses that induce heating in the mixture. Additionally, it will be appreciated that equipment temperatures, and residence times will be selected to minimize the amount of time that the admixture placed into the extruder spends under conditions of heating and/or shear stress so as to minimize the amount of Compound II, which is decomposed during formation of the composition, as discussed above. In general, extrusion processes in which heating is applied to the material extruded are termed “hot melt extrusion processes.” When compositions of the present invention are prepared using extrusion equipment, the extrudate thus provided can be in any convenient shape, for example, noodles, cylinders, bars, or the like. If desired, the extrudate can be further processed, for example by milling, to provide a particulate form of the composition.

As demonstrated by the Examples, the oral absorption of Compound II when formulated as a solid dispersion intermediate together with one or more pharmaceutically acceptable polymer, such as HPMC, together with optional surfactants, such as TPGS, is superior to formulations based on undispersed amorphous Compound II.

The relative amount of drug, polymer, and optional surfactant can vary widely. The optimal amount of the polymer, and optional surfactant can depend, for example, the hydrophilic lipophilic balance (HLB), melting point, and water solubility of the copolymer, and the surface tension of aqueous solutions of the surfactant, the properties of the drug, etc.

The compositions of the first solid dispersion formulation comprise an effective amount of Compound II, but comprise less than about 50% w/w of Compound II due to the relatively poor dissolution seen with formulations having greater than 50% w/w of Compound II. Thus, the concentration of Compound II can vary from about 0.1% to about 40.0%, from about 5.0% to about 35.0%, or from about 10% to about 30%, by weight based on the total combined weight of the drug substance, polymer, and optional surfactant (not including other excipients).

The concentration of the surfactant in the solid dispersion formulation can vary from about 2.0% to about 20%, or about 5% to about 15%, or about 10% by weight based on the total combined weight of the drug substance, polymer, and surfactant (not including other excipients).

The concentration of the pharmaceutically acceptable polymer in the solid dispersion formulation is added to the concentrations of the Compound II and surfactant to add up to 100%. The concentration can vary from about 50% to about 95% by weight based on the total combined weight of the drug substance, polymer, and optional surfactant, not including other excipients.

In embodiments, the solid dispersion formulation may comprise from between 5% to 50% of Compound II or a pharmaceutically acceptable salt thereof, 2.0% to about 20% surfactant, with the balance of the formulation being the pharmaceutically acceptable polymer.

### Blended Compositions

Embodiments of the invention relate to blended compositions that comprise Compound I, the solid dispersion formulation of Compound II, and optionally one or more of a diluent, disintegrant, salt, lubricant, and glidant. In all embodiments, all variables with respect to the solid dispersion formulations are as provided above.

In a first embodiment, Compound I is present in the blended composition in a concentration of from about 5% w/w to about 50% w/w. In particular instances, the first solid dispersion formulation is present in the blended composition in a concentration of from about 10% w/w to about 30% w/w, or about 20% w/w.

In a second embodiment, the solid dispersion formulation, comprising Compound II, is present in the blended composition in a concentration of from about 6% w/w to about 40% w/w. In particular aspects, the second solid dispersion formulation is present in the blended composition in a concentration of from about 10% w/w to about 16% w/w, or about 14% w/w.

In a third embodiment, the diluent in the blended composition is one or more pharmaceutically acceptable diluents selected from the group consisting of mannitol, microcrystalline cellulose, calcium carbonate, sodium carbonate, lactose, dicalcium phosphate, sodium phosphate, and starch, and combinations thereof. In particular aspects, the diluent is one or more selected from the group consisting of microcrystalline cellulose, mannitol, and dicalcium phosphate. In a particular instance, the diluent is a combination of mannitol and microcrystalline cellulose.

In a fourth embodiment, the diluent is present in the blended composition in a concentration of from about 3% w/w to about 58% w/w. In particular instances, the diluent is present in a concentration of from about 18% w/w to about 40% w/w, or about 28% w/w.

In a fifth embodiment, the disintegrant in the blended composition is selected from the group consisting of croscarmellose sodium, sodium starch glycolate, and crospovidone. In particular instances, the disintegrant is croscarmellose sodium.

In a sixth embodiment, the disintegrant is present in the blended composition in a concentration of from about 4% w/w to about 20% w/w. In particular instances, the disintegrant is present in a concentration of from about 7% w/w to about 15% w/w, or about 10% w/w.

In a seventh embodiment, an ionic salt may be present in the blended composition to further enhance the disintegration of the dosage form. The salt is selected from the group consisting of NaCl, KCl, CaCl₂, KH₂PO₄, NaH₂PO₄, K₂SO₄, NaHCO₃, K₂CO₃, and combinations thereof. In aspects, the salt in the blended composition is selected from the group consisting of NaCl, KCl, CaCl₂, and combinations thereof. In a particular instance, the salt is NaCl.

In an eighth embodiment, the salt is present in the blended composition in a concentration of from about 0% w/w to about 30% w/w. In particular instances, the salt is present in a concentration of from about 7% w/w to about 18% w/w, or about 10% w/w.

In a ninth embodiment, the lubricant in the blended composition is one or more pharmaceutically acceptable diluents selected from the group consisting of magnesium stearate, sodium stearyl fumarate, stearic acid, and glyceryl behenate. In a particular instance, the lubricant is a combination of magnesium stearate and sodium stearyl fumarate.

In a tenth embodiment, the lubricant is present in the blended composition in a concentration of from about 0.5% w/w to about 4% w/w. In particular instances, the lubricant is present in a concentration of from about 1% w/w to about 3% w/w, or about 2% w/w.

In an eleventh embodiment, the glidant in the blended composition is selected from the group consisting of starch, talc, magnesium stearate, and silicon dioxide, and combinations thereof. In a particular instance, the glidant is silicon dioxide.

In a twelfth embodiment, the glidant is present in the blended material in a concentration of from about 0% w/w to about 2% w/w. In particular instances, the glidant is present in a concentration of from about 0.1% w/w to about 1% w/w, or about 0.25% w/w.

A thirteenth embodiment is directed to a process for preparing a blended composition comprising the steps of: a) preparing a blended material by i) preparing a solid dispersion formulation comprising Compound II as described above by spray drying, extruding, milling, or other known or later-discovered process for making a solid dispersion formulation, ii) blending the solid dispersion formulation with one or more of a diluent, disintegrant, salt, lubricant, and glidant, and iii) optionally granulating, to produce the blended material; b) blending Compound I, the blended material, and optionally one or more of a diluent, disintegrant, salt, lubricant, and glidant together; c) optionally granulating the blend of step c), to produce a blended composition; and d) optionally further blending the blended composition of step d) with one or more extra-granular excipient(s) selected from diluents, disintegrants, salts, lubricants, and glidants. In aspects of this embodiment, blending a solid dispersion formulation with one or more other APIs, and or excipients may comprise blending alone, blending followed by granulation, or granulation followed by blending with the excipients. Granulation, as used herein, includes all known, and later-developed methods of creating granules.

In aspects of these embodiments, the diluents, disintegrants, salts, lubricants, and/or glidants are as described above. The diluents, disintegrants, salts, lubricants, and/or glidants may be present in the concentrations described above.

### Oral Dosage Forms

In a fourteenth embodiment, the blended composition is formulated as a tablet or as a capsule.

A fifteenth embodiment of the invention is directed to a process for preparing a solid pharmaceutical composition comprising the steps of: a) preparing a blended composition as described above in the thirteenth embodiment; b) compressing the blended composition into a tablet or filling into a capsule. In aspects of the fifteenth embodiment, the tablet is optionally film-coated; in further aspects, the tablet or capsule is optionally photo-shielded, for example by use of a blister packaging.

In aspects of these embodiments, the diluents, disintegrants, salts, lubricants, and/or glidants are as described above with respect to blended compositions. The diluents, disintegrants, salts, lubricants, and/or glidants may be present in the concentrations described above with respect to blended compositions.

Pharmaceutical compositions intended for oral use may be prepared from the solid dispersion formulations, and blended materials described above in accordance with the methods described herein, and other methods known to the art for the manufacture of pharmaceutical compositions. Such compositions may further contain one or more agents selected from the group consisting of sweetening agents, flavoring agents, coloring agents, and preserving agents in order to provide pharmaceutically elegant and palatable preparations.

Tablets may contain the active ingredient in admixture with non-toxic pharmaceutically acceptable excipients that are suitable for the manufacture of tablets. These excipients may be for example, inert diluents, granulating, and disintegrating agents, binding agents, glidants, lubricating agents, and antioxidants, for example, propyl gallate, butylated hydroxyanisole, and butylated hydroxy toluene. The tablets may be uncoated or they may be film coated to modify their appearance or may be coated with a functional coat to delay disintegration, and absorption in the gastrointestinal tract, and thereby provide a sustained action over a longer period.

Compositions for oral use may also be presented as capsules (e.g., hard gelatin) wherein the active ingredient is mixed with an inert solid diluent, for example, calcium carbonate, calcium phosphate or starch, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent, and one or more preservatives. In certain embodiments of the invention, the pharmaceutical compositions of the invention include a diluent system, disintegrant, salt, lubricant, glidant, and filmcoat, at concentrations of from about 3% w/w to about 58% w/w, from about 4% w/w to about 20% w/w, from about 4% w/w to about 20% w/w, from about 0.5% w/w to about 4% w/w, from about 0% w/w to about 2% w/w, and from about 1% w/w to about 5% w/w respectively, or at from about 18% w/w to about 40% w/w, from about 7% w/w to about 15% w/w, from about 7% w/w to about 18% w/w, from about 1.0% w/w to about 3.0%, from about 0.1% w/w to about 1.0% w/w, and from about 2.0% w/w to about 4.0% w/w, respectively. In certain embodiments, the solid dispersion formulations are blended with a diluent, one or more disintegrating agents, lubricants, and glidants. An exemplary blended composition or oral dosage form includes mannitol, microcrystalline cellulose, croscarmellose sodium, sodium chloride, colloidal silica, sodium stearyl fumarate, and magnesium stearate.

The disintegrant may be present in a concentration from about 4% w/w to about 20% w/w or from about 7% w/w to about 15% w/w. A salt may be also present, which may be sodium chloride, potassium chloride or a combination thereof. The combination of salts and disintegrant is present at a concentration from about 5% w/w to about 35% w/w of the final pharmaceutical composition.

The blended compositions may be roller compacted or wet granulated to densify, and/or reduce the risk of segregation of components during subsequent handling (e.g., compression into tablets). Granulation steps can also be used to minimize the impact of raw material property variability (e.g., excipient particle size) on subsequent processing (e.g., tablet compression) and ultimate product performance. Lubrication is typically performed prior to roller compaction and tablet compression to reduce the tendency of material to adhere to compression surfaces (e.g., tablet tooling). In particular embodiments, the lubricant system is a combination of sodium stearyl fumarate and magnesium stearate. These methods can be carried out by those skilled in the art. See, e.g., Ansel, Introduction to Pharmaceutical Dosage Forms, Seventh Edition, 1999.

To prepare the pharmaceutical compositions of the invention, the blended composition is compressed into an oral dosage form such as tablets. Tablets can be prepared with a variety of possible shapes (ellipsoidal, capsule, biconvex round, etc.). The powder can also be encapsulated in capsule dosage forms (e.g., using hard gelatin capsules or capsules fabricated from hydroxypropyl methylcellulose). Techniques suitable for preparing solid oral dosage forms of the present invention are described in Remington's Pharmaceutical Sciences, 18th edition, edited by A. R. Gennaro, 1990, Chapter 89, and in Remington—The Science, and Practice of Pharmacy, 21st edition, 2005, Chapter 45. In certain embodiments, the first solid dispersion formulation is present in an amount of from about 6% w/w to about 30% w/w of the pharmaceutical composition or from about 10% w/w to about 16% w/w of the final pharmaceutical composition, and the second solid dispersion formulation is present in an amount of from about 6% w/w to about 30% w/w of the pharmaceutical composition or from about 10% w/w to about 18% w/w of the final pharmaceutical composition.

As demonstrated by the examples, a solid dispersion formulation of Compound II showed robust pharmacokinetic performance when dosed with pH-raising medication. When the combination formulation containing Compound I and the solid dispersion formulation of Compound II was prepared as an oral dosage form as described herein, it was found to maintain the pharmacokinetic performance of each of Compound I and Compound II, and to provide robust absorption regardless of gastric pH modulation due to the use of, for example, H2-receptor antagonists or proton-pump inhibitors.

### Additional Combination Dosage Forms

Additional embodiments include combination regimens, comprising the fixed dose combinations as described above, and one or more additional drug substance(s). For combination regimens, other drug substance(s) can be added to the solid dispersion or the tablet formulation, either in a crystalline form, neat amorphous form, or as a solid dispersion. In particular combination regimens, one or more additional drug substance(s) are formulated either as pure APIs or as solid dispersion formulations, and Compound I, the solid dispersion formulation of Compound II, and any additional drug substance(s), however formulated, are combined into a blended composition, and provided as a dosage form that may be a tablet or capsule. Additional extra-granular components may also be combined into the blended composition, such as diluents, disintegrants, salts, lubricants, and glidants, as described above.

Exemplary drug substances that may be included as the additional drug substance(s) include, but are not limited to, HCV protease inhibitors, HCV polymerase inhibitors, HCV NS4A inhibitors, HCV NS5A inhibitors, and HCV NS5b inhibitors.

HCV protease inhibitors include, but are not limited to, those disclosed in U.S. Pat. Nos. 8,080,654; 7,973,040; 8,828,930; 8,927,569; 7,879,797; 7,470,664; 8,216,999; 8,377,873; 8,278,322; 8,138,164; 8,377,874; 8,309,540; 8,591,878; 7,494,988; 7,485,625; 7,795,250; 7,449,447; 7,442,695; 7,425,576; 7,342,041; 7,253,160; 7,244,721; 7,205,330; 7,192,957; 7,186,747; 7,173,057; 7,169,760; 7,012,066; 6,914,122; 6,911,428; 6,894,072; 6,846,802; 6,838,475; 6,800,434; 6,767,991; 5,017,380; 4,933,443; 4,812,561, and 4,634,697; U.S. Patent Application Publication Nos. US2014/0057836, US2013/0178413, US2010/0099695, US2014/0296136, US2002/0068702, US2002/0160962, US2005/0119168, US2005/0176648, US2005/0209164, US2005/0249702, and US2007/0042968; and PCT International Patent Application Publication Nos. WO2014/025736, WO2009/010804, WO2010/011566, WO2011/014487, WO2006/119061, WO2007/015855, WO2007/015787, WO2007/016441, WO2007/131966, WO2007/148135, WO2008/057209, WO2008/051475, WO2008/057208, WO2008/051514, WO2009/108507, WO2008/051477, WO2012/040040, Wo2013/074386, WO03/006490, WO03/087092, WO04/092161, and WO08/124148.

HCV protease inhibitors also include, but are not limited to, boceprevir, narlaprevir, vaniprevir, grazoprevir, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex), and PHX1766 (Phenomix).

Further examples of HCV protease inhibitors include, but are not limited to, those disclosed in Landro et al., 36(31) BIOCHEMISTRY 9340-9348 (1997); Ingallinella et al., 37(25) BIOCHEMISTRY 8906-8914 (1998); Llinàs-Brunet et al., 8(13) BIOORG. MED. CHEM. LETT. 1713-1718 (1998); Martin et al., 37(33) BIOCHEMISTRY 11459-11468 (1998); Dimasi et al., 71(10) J. VIROL. 7461-7469 (1997); Martin et al., 10(5) PROTEIN ENG. 607-614 (1997); Elzouki et al., 27(1) J. HEPAT. 42-48 (1997); 9(217) BIOWORLD TODAY 4 (Nov. 10, 1998); U.S. Patent Application Publication Nos. US2005/0249702 and US 2007/0274951; and PCT International Patent Application Publication Nos. WO98/14181, WO98/17679, WO98/22496, WO99/07734, and WO05/087731.

HCV polymerase inhibitors include, but are not limited to, those disclosed in U.S. Pat. No. 8,183,216; U.S. Patent Application Publication Nos. US2011/0306573, US2014/0206640, and US2014/0161770; and PCT International Patent Application Publication Nos. WO09/040269, WO2013/177219, WO2014/058801, WO2014/062596, and WO2012/142085.

HCV polymerase inhibitors include, but are not limited to, VP-19744 (WyethNiroPharma), PSI-7851 (Pharmasset), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716 (ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., 7(4) CURRENT OPINION IN DRUG DISCOVERY, AND DEVELOPMENT 446 (2004); Tan et al., 1 NATURE REVIEWS 867 (2002); and Beaulieu et al., 5 CURRENT OPINION IN INVESTIGATIONAL DRUGS 838 (2004).

HCV NS4A inhibitors include, but are not limited to, those disclosed in U.S. Pat. Nos. 7,476,686 and 7,273,885; U.S. Patent Application Publication No. US2009/0022688; and PCT International Patent Application Publication Nos. WO2006/019831 and WO2006/019832. Additional HCV NS4A inhibitors include, but are not limited to, AZD2836 (Astra Zeneca) and ACH-806 (Achillon Pharmaceuticals, New Haven, Conn.).

HCV NS5A inhibitors include, but are not limited to, those disclosed in U.S. Pat. Nos. 8,871,759 and 8,609,635; U.S. Patent Application Publication No. US2014/0371138; and PCT International Patent Application Publication Nos. WO2014/110705 and WO2014/110706.

HCV NS5B inhibitors include, but are not limited to, those disclosed in U.S. Patent Application Publication No. US2012/0328569; and PCT International Patent Application Publication Nos. WO2010/111483, WO2011/106992, WO2011/106985, and WO2011/106929.

A further embodiment of the invention is directed to a process for preparing a solid pharmaceutical composition comprising the steps of: a) preparing a blended material by i) preparing a solid dispersion formulation comprising Compound II as described above by spray drying, extruding, milling or other known or later-discovered process for making a solid dispersion formulation, ii) blending the solid dispersion formulation with one or more of a diluent, disintegrant, salt, lubricant, and glidant, and iii) granulating, such as by roller compaction to produce the first blended material; b) preparing one or more blended material(s) by i) preparing one or more additional solid dispersion formulation(s) comprising one or more additional APIs by spray drying, extruding, milling or other known or later-discovered process for making a solid dispersion formulation, ii) blending the one or more additional solid dispersion formulation with one or more of a diluent, disintegrant, salt, lubricant, and glidant, and iii) granulating, such as by roller-compaction to produce the one or more additional blended material; c) mixing Compound I, the blended material, and one or more additional blended material together; d) mixing the blend of step c) with a lubricant, and optionally one or more of a diluent, disintegrant, salt, and glidant together; and e) optionally granulating the blend of step d), to produce a blended composition; f) compressing the particles into a tablet or filling into a capsule. In addition, the tablet is optionally film-coated; in further aspects, the tablet or capsule is optionally photo-shielded, for example by use of a blister packaging.

The following examples serve only to illustrate the invention, and its practice. The examples are not to be construed as limitations on the scope or spirit of the invention.

In addition, the following abbreviations are used throughout this specification, and in the Examples. Each of these terms has the meaning listed below.

### ABBREVIATIONS

AUC_{0-∞} Area under the concentration time curve from time zero to infinity
AUC₀₋ₗₐₛₜ Area under the concentration time curve from time zero to last dose
AUC₀₋₂₄. Area under the concentration time curve from time zero to 24 hours
bar Metric unit of pressure, 1 bar=100,000 Pascal
CI Confidence interval
Cₘₐₓ Maximum concentration (specifically of a drug)
C₂₄ Maximum concentration over 24 hours (specifically of a drug)
f³ Cubic feet, blender capacity
g Gram(s)
GM Geometric mean
GMR Geometric mean ratio
HPMC Hydroxypropylmethyl cellulose
HPMCAS Hydroxypropylmethyl cellulose acetate succinate
hr Hour(s)
kg Kilogram(s)
kP, kgf Kilopond, a non-standard gravitational unit of force, also kilogram-force; 1 kP=9.80665 Newtons
L Liter
mg Milligram
min Minute(s)
mL Milliliter
mm Millimeter
MPa Mega Pascal
nM Nanomolar
PSI, psi Pounds per square inch [gauge], 1 Pascal=0.000145037738007 psi
RPM Revolutions per minute
SLS Sodium lauryl sulfate
TPGS Vitamin E polyethylene glycol succinate
w/w, % w/w Percentage by weight (i.e., grams of solute in 100 g of solution)
μM Micromolar

### EXAMPLES

### Example 1: Direct Compression Tablet Formulation of Compound I

Compound I may be prepared as disclosed in General Method F of PCT International Patent Application Publication Nos. WO2013/177219 and WO2014/058801.

Formulation 1 is a direct-compressed tablet formulation (Table 1) containing 150 mg of Compound I in which crystalline Compound I is combined with microcrystalline cellulose, mannitol, crospovidone, and magnesium stearate, and compressed into tablets. To produce the tablets of Formulation 1, Compound I, and the inert excipients, with the exception of the magnesium stearate, were weighed, optionally passed through a screen (Quadro Comil, 610 μm screen, round impeller, 450 rpm), and blended together (3 ft³ V-blender, 250 revolutions). The magnesium stearate was weighed, optionally passed through a screen (No. 30 Mesh), and blended with the other components (3 ft³ V-blender, 50 revolutions). The lubricated blend was then compressed into tablets on a rotary tablet press. The compression parameters were adjusted to achieve acceptable tablet hardness, and friability, and a disintegration time of not more than 15 min.

TABLE 1
Composition of Formulation 1

| Component | Amount (mg/tablet) |
|---|---|
| Compound I | 150 |
| Microcrystalline cellulose | 150 |
| Mannitol | 270 |
| Crospovidone | 24 |
| Magnesium stearate | 6 |
| Total | 600 |

### Example 2: Conventional Wet-Granulated Formulation of Compound II

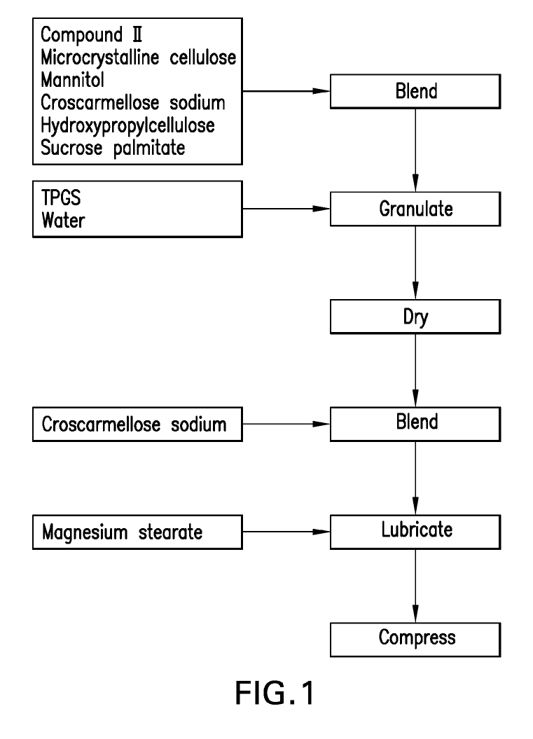

Conventional Formulation 2 is a conventional wet-granulated tablet formulation of Compound II in which Compound II is formulated as a pure amorphous API (Table 2). 60 mg of Compound II is blended with microcrystalline cellulose, mannitol, hydroxypropylcellulose, sucrose palmitate, and a portion of the croscarmellose sodium, added to the bowl of a high-shear granulator, and granulated with a solution of 20% TPGS in water. The resulting granules are dried, milled through a screen with an opening size of approximately 0.8 mm, blended with the remaining croscarmellose sodium, lubricated with the magnesium stearate, and compressed into tablets. The tablet weight was 200 mg using 10/32 in standard round concave tooling, and the compression parameters were adjusted to achieve a tablet tensile strength in the range 100-200 MPa. FIG. 1 outlines the process used to make Conventional Formulation 2.

TABLE 2
Composition of Conventional Formulation 2

| Component | Amount (mg/tablet) |
|---|---|
| Compound II | 60.0 |
| Microcrystalline cellulose | 47.5 |
| Mannitol | 47.5 |
| Hydroxypropylcellulose | 6.0 |
| Croscarmellose sodium | 18.0 |
| TPGS | 16.0 |
| Sucrose palmitate | 4.0 |
| Magnesium stearate | 1.0 |
| Total | 200.0 |

The oral absorption obtained from Conventional Formulation 2 was determined in a preclinical pharmacokinetic study conducted in beagle dogs. The results are shown in Table 3.

TABLE 3
Summary of PK Results (Mean ± SE) for 60 mg Doses of Compound II
Administered as Conventional Formulation 2 or as Control (Conventional
Dry-Filled Capsule) Formulation to Pentagastrin-Pre-Treated Male
Beagle Dogs

| | Conventional Formulation 2 | Control |
|---|---|---|
| AUC_{0-∞} (nM · h) | 9,205 ± 409 | 11,556 ± 2210 |
| Cₘₐₓ (nM) | 1,844 ± 68 | 1,705 ± 140 |

### Example 3: HPMC-TPGS Formulation of Compound II

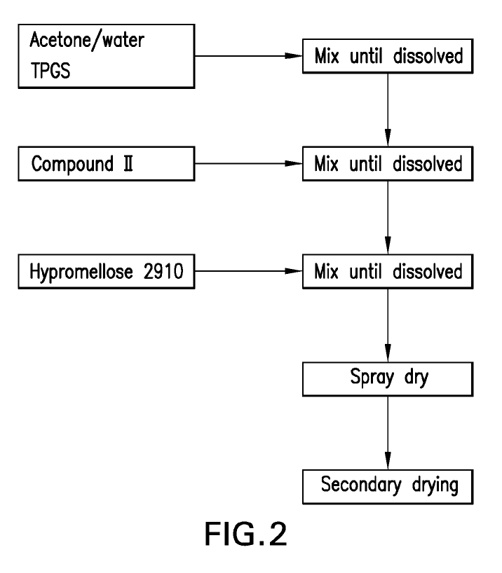

Solid Dispersion Formulation 3 is used in a tablet composition, Tablet Formulation 1, containing a solid dispersion of Compound II as shown in Table 4. The solid dispersion was prepared from a solution comprising Compound II, TPGS, and HPMC by spray drying from an acetone/water solvent system, as shown in FIG. 2. The solid dispersion was prepared by spray-drying a solution comprising Compound II, HPMC, and TPGS in an acetone/water solvent system. The spray-drying solution was prepared such that it contained 10% solids in solution. This solution was then spray-dried using a NIRO PSD-1 spray dryer with a pressure nozzle to produce the spray-dried particles. The spray-dried particles were flash-dried in a chamber that can contain an inert heated gas (e.g., nitrogen). Heated nitrogen was supplied to the spray dryer at an inlet temperature sufficient to maintain a 40° C. outlet temperature and a gas flow rate of approximately 1850 g/min. The spray drying solution flow rate was 120 g/min, with a nozzle pressure of approximately 240 psi using an SK80-16 pressure nozzle. The particles thus produced were collected using a cyclone. Typically, a secondary-drying operation is used to further dry the spray dried particles collected as described above. Nitrogen or air may be used to facilitate drying, using either tray dryers or agitated dryers. In this case, the solid dispersion of Formulation 3 was secondary-dried using a tray-drier, at drying conditions of 21° C. and 45% RH.

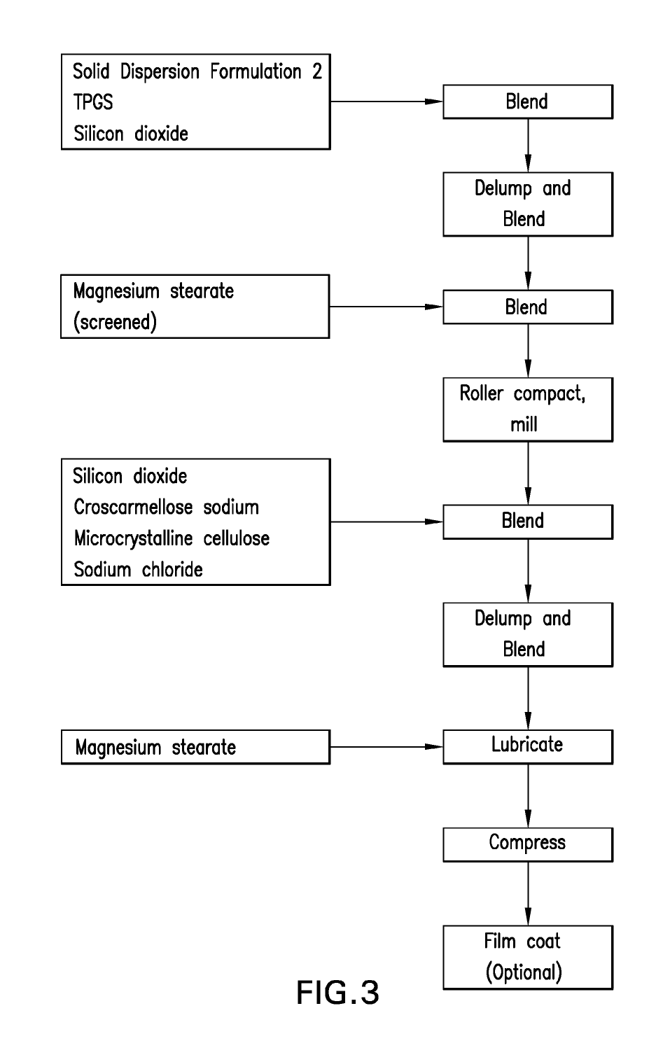

The resulting solid dispersion was blended with portions of the silicon dioxide and magnesium stearate, and roller-compacted. The roller-compacted material was further blended with the microcrystalline cellulose, sodium chloride, croscarmellose sodium, and the remaining silicon dioxide, blended, lubricated with the remaining magnesium stearate, and compressed into tablets on a rotary tablet press. The compression parameters were adjusted to achieve acceptable hardness, friability, and disintegration time. The process for producing Tablet Formulation 1 is illustrated in FIG. 3.

TABLE 4
Composition of Tablet Formulation 1

| | |
|---|---|
| Component | Amount (mg/tablet) |

Solid Dispersion Formulation 3

| | |
|---|---|
| Compound II | 60 |
| Hypromellose 2910 | 210 |
| TPGS | 30 |

Remaining Components

| | |
|---|---|
| Microcrystalline cellulose | 191.82 |
| Sodium chloride | 60 |
| Croscarmellose sodium | 36 |
| Silicon dioxide | 9.15 |
| Magnesium stearate | 3.03 |
| Total | 600 |

### Example 4: HPMCAS Formulation of Compound II

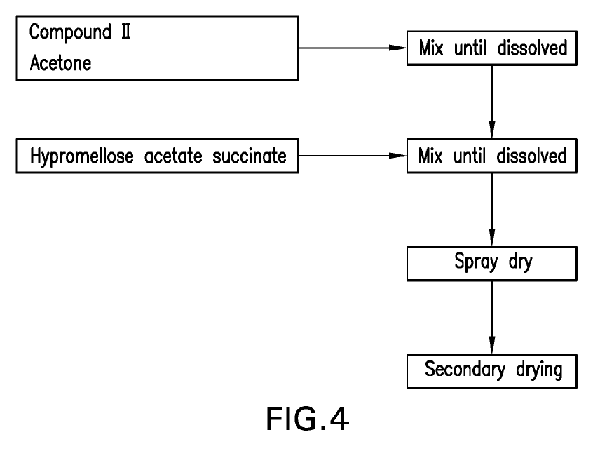

Solid Dispersion Formulation 4 is used in a tablet composition, Tablet Formulation 2, containing a solid dispersion of Compound II as shown in Table 5. The solid dispersion was prepared from a solution comprising Compound II and HPMCAS by spray drying from acetone, as shown in FIG. 4. The solid dispersion was prepared by spray-drying a solution comprising Compound II and HPMCAS in acetone. The spray-drying solution was prepared such that it contained 10% solids in solution. This solution was then spray-dried using a NIRO PSD-1 spray dryer with a pressure nozzle to produce the spray-dried particles. The spray-dried particles were flash-dried in a chamber that can contain an inert heated gas (e.g., nitrogen). Heated nitrogen was supplied to the spray dryer at an inlet temperature sufficient to maintain a 35° C. outlet temperature and a gas flow rate of approximately 1850 g/min. The spray drying solution flow rate was 195 g/min, with a nozzle pressure of approximately 305 psi using a Steinen A75 pressure nozzle. The particles thus produced were collected using a cyclone. Solid Dispersion Formulation 4 was secondary-dried using a tray-drier, at drying conditions of 21° C. and 45% RH.

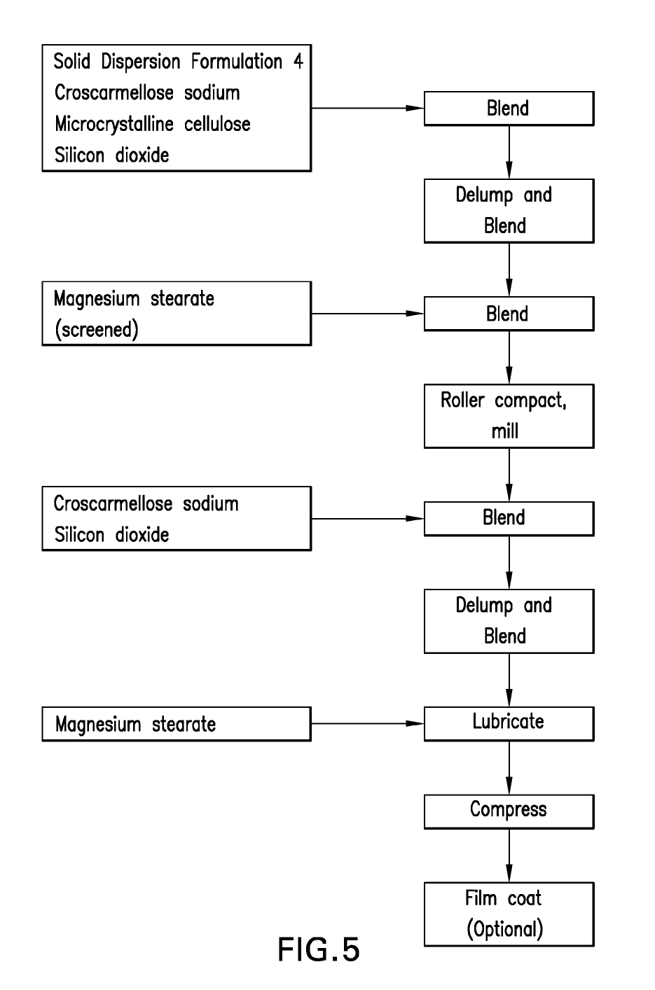

The resulting solid dispersion was blended with the lactose, microcrystalline cellulose, and portions of the croscarmellose sodium, silicon dioxide, and magnesium stearate and roller-compacted. The roller-compacted material was further blended with the remaining croscarmellose sodium and silicon dioxide, blended, lubricated with the remaining magnesium stearate, and compressed into tablets on a rotary tablet press. The compression parameters were adjusted to achieve acceptable tablet hardness, friability, and disintegration time. The procedure for preparing Tablet Formulation 2 is illustrated by FIG. 5.

TABLE 4
Composition of Tablet Formulation 2

| | |
|---|---|
| Component | Amount (mg/tablet) |

Solid Dispersion Formulation 4

| | |
|---|---|
| Compound II | 60 |
| Hypromellose Acetate Succinate | 240 |

Remaining Components

| | |
|---|---|
| Microcrystalline cellulose | 81.25 |
| Lactose | 81.25 |
| Croscarmellose Sodium | 30 |
| Silicon Dioxide | 5 |
| Magnesium Stearate | 2.5 |
| Total | 500 |

The pharmacokinetics of Compound II from Tablet Formulation 1 and Tablet Formulation 2 were evaluated in a human clinical trial in healthy normal subjects, under fasted, and fed conditions, and following administration of a proton pump inhibitor, famotidine. Table 5 shows the pharmacokinetics of Compound II when administered as Tablet Formulation 1, and Table 6 shows the pharmacokinetics of Compound II when administered as Tablet Formulation 2. In each case, the pharmacokinetics of Compound II from these formulations were compared to the pharmacokinetics of Compound II when administered as a dry-filled capsule control formulation of pure amorphous Compound II mixed with conventional pharmaceutical excipients.

TABLE 5
Pharmacokinetics of Compound II in human subjects after
administration as Tablet Formulation 1, compared to control
formulation (Geometric Mean data (% CV))

| | AUC_{0-24 hr} | | |
|---|---|---|---|
| Treatment | (nM * hr) | Cₘₐₓ (nM) | C₂₄ (nM) |
| Control Dry-Filled Capsule | 1297 (50.2) | 145 (51.0) | 23.9 (55.1) |
| (6 × 10 mg) | | | |
| Tablet Formulation 1 with | 1193 (37.4) | 121 (40.4) | 22.4 (44.7) |
| Famotidine | | | |
| Tablet Formulation 1, Fasted | 1391 (54.0) | 148 (62.2) | 25.6 (51.8) |
| Tablet Formulation 1, Fed | 460 (48.6) | 41.4 (63.3) | 10.7 (42.9) |

TABLE 6
Pharmacokinetics of Compound II in human subjects after administration
as Tablet Formulation 2, compared to control formulation (Geometric
Mean data (% CV))

| | AUC_{0-24 hr} | | |
|---|---|---|---|
| Treatment | (nM * hr) | Cₘₐₓ (nM) | C₂₄ (nM) |
| Control Dry-Filled Capsule | 1048 (66.3) | 119 (75.6) | 19.2 (67.3) |
| (6 × 10 mg) | | | |
| Tablet Formulation 2 | 986 (45.6) | 103 (51.0) | 18.4 (45.6) |
| with Famotidine | | | |
| Tablet Formulation 2, Fasted | 820 (51.5) | 86.3 (53.4) | 16.7 (50.1) |
| Tablet Formulation 2, Fed | 303 (50.2) | 22.9 (55.9) | 8.22 (61.6) |

These results showed that Tablet Formulation 1 gave slightly higher exposures of Compound II compared to the control formulation of pure amorphous Compound II, while Tablet Formulation 2 gave slightly lower exposures compared to the control. Under fed conditions both Tablet Formulation 1 and Tablet Formulation 2 exhibited lower exposures of Compound II compared to the control formulation.

### Example 5: Fixed-Dose Combination Formulation of Compound I and Compound II

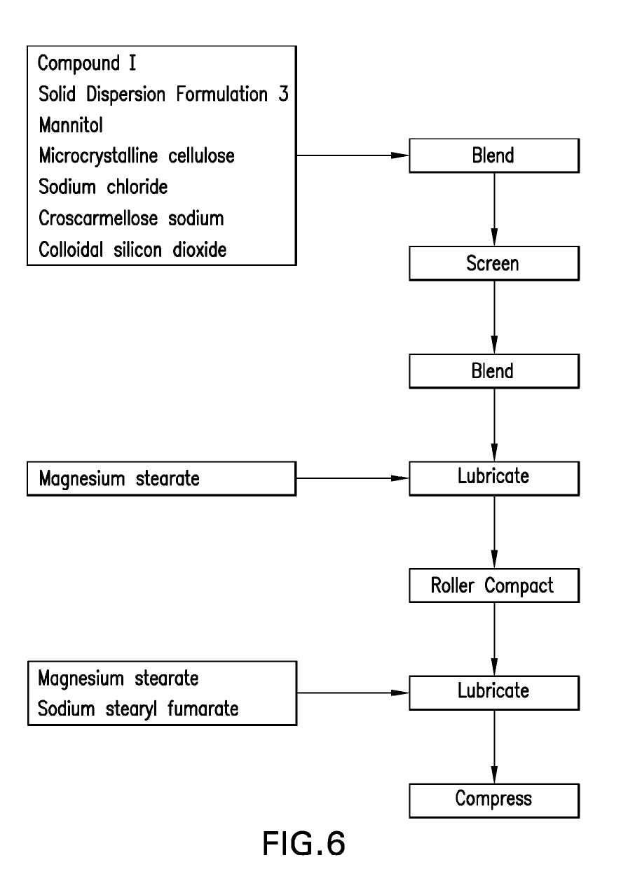

A fixed-dose combination Tablet Formulation 3 comprising Compound I and Solid Dispersion Formulation 3 (the solid dispersion formulation of Compound II that was prepared according to Example 3 above). Table 7 provides the composition of Tablet Formulation 3. FIG. 6 illustrates the process for preparing the tablets of Table 7. The solid dispersion of Compound II was combined with Compound I and the mannitol, microcrystalline cellulose, croscarmellose sodium, sodium chloride, silicon dioxide, and a portion of the magnesium stearate. These components were blended in a bin blender (Bohle 3L bin, 300 total revolutions) and then roller-compacted using a Gerteis Mini-Pactor roller compactor (knurled rolls, 8.1 kN/cm roll force, Conidur 1 mm mill screen). The resulting granulation was blended with the remaining magnesium stearate and sodium stearyl fumarate (Bohle 3L bin, 60 revolutions), and compressed into 1500 mg image tablets using a single station tablet press. The compression parameters were controlled to achieve tablet hardness in the range 50 to 65 kP.

TABLE 7
Fixed-Dose Combination Tablet Formulation 3

| Component | Amount (mg/tablet) |
|---|---|
| Compound I | 450.0 |

Spray Dried Intermediate of Compound II - Solid Dispersion
Formulation 3

| | |
|---|---|
| Compound II | 60.00 |
| Hypromellose 2910 | 210.00 |
| TPGS | 30.00 |

Remaining Components

| | |
|---|---|
| Mannitol | 139.82 |
| Microcrystalline cellulose | 279.64 |
| Sodium Chloride | 150.0 |
| Silicon Dioxide, Colloidal | 0.55 |
| Magnesium Stearate | 15.00 |
| Sodium stearyl fumarate | 15.00 |
| Total Coated Tablet Weigh | 1500.00 |

### Example 6: Fixed-Dose Combination Formulation of Compound I and Compound II

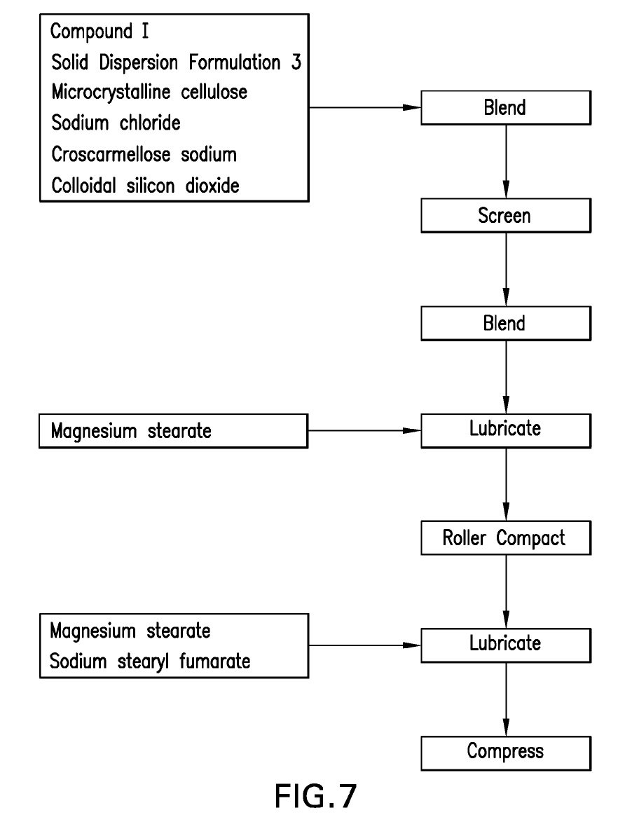

A fixed-dose combination Tablet Formulation 4 comprising Compound I and Solid Dispersion Formulation 3 (the solid dispersion formulation of Compound II that was prepared according to Example 3 above). Table 8 provides the composition of Tablet Formulation 4. FIG. 7 illustrates the process for preparing the tablets of Table 8. The solid dispersion of Compound II was combined with Compound I and the microcrystalline cellulose, croscarmellose sodium, sodium chloride, silicon dioxide, and a portion of the magnesium stearate. These components were blended in a Turbula Mixer, and then granulated by producing simulated roller compacted ribbons of target tensile strength 0.6 to 1.0 MPa, which were then milled through a 1 mm mill screen. The resulting granulation was blended with the remaining magnesium stearate and sodium stearyl fumarate (Turbula Mixer), and compressed into 1500 mg tablets using a single station tablet press. The compression parameters were controlled to achieve tablet hardness in the range 50 to 65 kP.

TABLE 8
Fixed-Dose Combination Tablet Formulation 4

| Component | Amount (mg/tablet) |
|---|---|
| Compound I | 450.0 |

Spray Dried Intermediate of Compound II - Solid Dispersion
Formulation 3

| | |
|---|---|
| Compound II | 60.00 |
| Hypromellose 2910 | 210.00 |
| TPGS | 30.00 |

Remaining Components

| | |
|---|---|
| Microcrystalline cellulose | 419.45 |
| Sodium Chloride | 150.0 |
| Croscarmellose sodium | 150.0 |
| Silicon Dioxide, Colloidal | 0.55 |
| Magnesium Stearate | 15.00 |
| Sodium stearyl fumarate | 15.00 |
| Total Coated Tablet Weigh | 1500.00 |

It will be appreciated that various of the above-discussed, and other features, and functions, or alternatives thereof, may be desirably combined into many other different systems or applications. Also that various presently unforeseen or unanticipated alternatives, modifications, variations or improvements therein may be subsequently made by those skilled in the art, which are also intended to be encompassed by the following claims.

1. A blended composition comprising
(a) (2R)-isopropyl 2-(((((2R,3R,4R,5R)-4-chloro-5-(2,4-dioxo-3,4-dihydropyrimidin-1(2H)-yl)-3-hydroxy-4-methyltetrahydrofuran-2-yl)methoxy)(phenoxy) phosphoryl)amino)propanoate (Compound I): 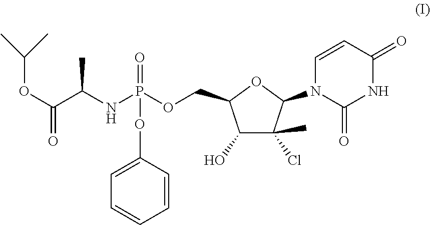 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]
or a pharmaceutically acceptable salt thereof; and
(b) a solid dispersion formulation, which comprises
(i) dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate (Compound II): 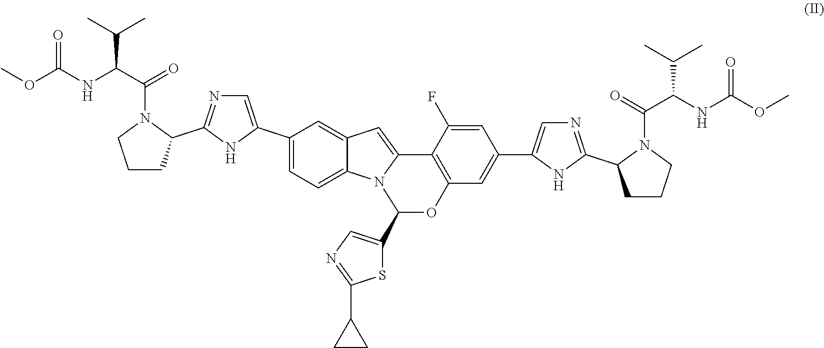
or a pharmaceutically acceptable salt thereof;
(ii) one or more pharmaceutically acceptable polymers or a mixture thereof; and
(iii) optionally one or more pharmaceutically acceptable surfactants or a mixture thereof; and
wherein Compound II, and the one or more surfactants, if present, are dispersed in a polymer matrix formed by the one or more pharmaceutically acceptable polymers; and
(c) optionally one or more of a diluent, disintegrant, salt, lubricant, and glidant.

2. The blended composition according to claim 1, wherein Compound I is substantially crystalline, and Compound II is substantially amorphous.

3. The blended composition according to claim 1, wherein
a) Compound I is present in a amount of from about 5% w/w to about 50% w/w; and
b) in the solid dispersion formulation,
i) Compound II is present in a concentration of from about 5% w/w to about 50% w/w, relative to the total combined weight of the solid dispersion formulation,
ii) the one or more pharmaceutically acceptable polymers or a mixture thereof is present in a concentration of from about 50% w/w to about 95% w/w, relative to the total combined weight of the solid dispersion formulation, and
iii) the one or more pharmaceutically acceptable surfactants is present in a concentration of from about 2% w/w to about 20% w/w, relative to the total combined weight of the solid dispersion formulation.

4. The blended composition according to claim 3, wherein
a) Compound I is present in a amount of from about 10% w/w to about 30% w/w; and
b) in the solid dispersion formulation,
i) Compound II is present in a concentration of from about 10% w/w to about 40% w/w, relative to the total combined weight of the solid dispersion formulation,
ii) the one or more pharmaceutically acceptable polymers or a mixture thereof is present in a concentration of from about 50% w/w to about 90% w/w, relative to the total combined weight of the solid dispersion formulation, and
iii) the one or more pharmaceutically acceptable surfactants is present in a concentration of from about 5% w/w to about 15% w/w, relative to the total combined weight of the solid dispersion formulation.

5. The blended composition according to claim 4, wherein
a) Compound I is present in a amount of about 20% w/w; and
b) in the solid dispersion formulation,
i) Compound II is present in a concentration of about 20% w/w, relative to the total combined weight of the solid dispersion formulation,
ii) the one or more pharmaceutically acceptable polymers or a mixture thereof is present in a concentration of about 70% w/w, relative to the total combined weight of the solid dispersion formulation, and
iii) the one or more pharmaceutically acceptable surfactants is present in a concentration of about 10% w/w, relative to the total combined weight of the solid dispersion formulation.

6. The blended composition according to claim 1, wherein in the solid dispersion formulation, the one or more pharmaceutically acceptable polymers or a mixture thereof is selected from the group consisting of cellulosic polymers.

7. The blended composition according to claim 6, wherein in the solid dispersion formulation, the one or more pharmaceutically acceptable polymers or a mixture thereof is HPMC.

8. The blended composition according to claim 1, wherein in the solid dispersion formulation, the one or more pharmaceutically acceptable surfactant is present, and is vitamin E TPGS.

9. The blended composition according to claim 1, further comprising one or more excipient selected from the group consisting of diluents, granulating agents, disintegrants, lubricants, glidants, sweetening agents, flavoring agents, coloring agents, preserving agents, binding agents, and antioxidants.

10. An oral dosage form comprising the blended composition according to claim 1.

11. The oral dosage form according to claim 10, wherein the oral dosage form is a tablet or a capsule.

12. The oral dosage form according to claim 11, wherein the oral dosage form is a tablet, and wherein the tablet is film-coated.

13. A process for preparing a blended composition, comprising
1) preparing a blended material by
a) preparing a solid dispersion formulation by spray drying, extruding or milling to form particles, said solid dispersion formulation comprising
(i) dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate (Compound II): 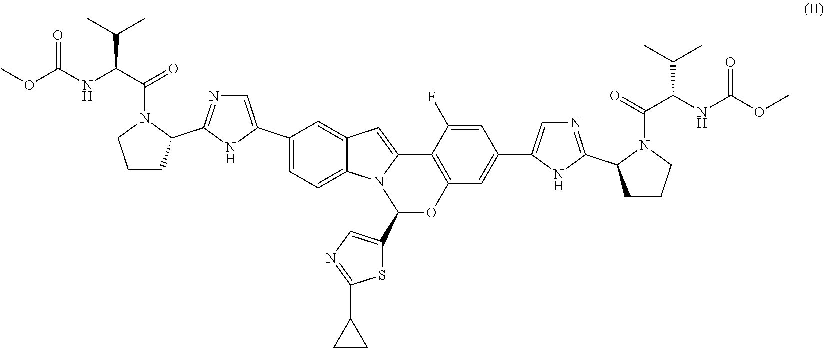
or a pharmaceutically acceptable salt thereof;
(ii) one or more pharmaceutically acceptable polymers or a mixture thereof; and
(iii) optionally one or more pharmaceutically acceptable surfactants or a mixture thereof; and
wherein Compound II, and the one or more surfactants are dispersed in a polymer matrix formed by the one or more pharmaceutically acceptable polymers; and
b) optionally blending the solid dispersion formulation with one or more of a diluent, disintegrant, salt, lubricant, and glidant, and
c) optionally granulating to form a second blended material; and
2) blending Compound I, the blended material, and optionally one or more of a diluent, disintegrant, salt, lubricant, and glidant to provide a blended composition, wherein Compound I is (2R)-isopropyl 2-(((((2R,3R,4R,5R)-4-chloro-5-(2,4-dioxo-3,4-dihydropyrimidin-1(2H)-yl)-3-hydroxy-4-methyltetrahydrofuran-2-yl)methoxy)(phenoxy) phosphoryl)amino)propanoate: 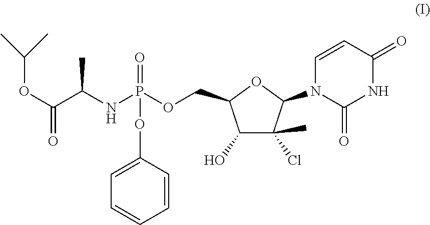 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]
or a pharmaceutically acceptable salt thereof.

14. A process for preparing an oral dosage form, comprising
1) preparing a blended material by
a) preparing a solid dispersion formulation by spray drying, extruding or milling to form particles, said solid dispersion formulation comprising
(i) dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate (Compound II): 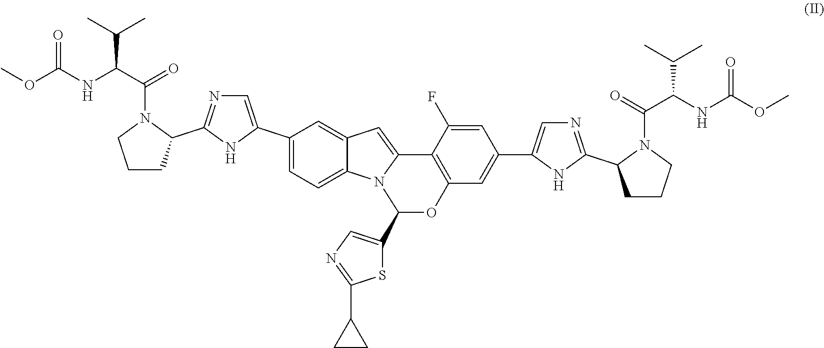
or a pharmaceutically acceptable salt thereof;
(ii) one or more pharmaceutically acceptable polymers or a mixture thereof; and
(iii) optionally one or more pharmaceutically acceptable surfactants or a mixture thereof; and
wherein Compound II, and the one or more surfactants are dispersed in a polymer matrix formed by the one or more pharmaceutically acceptable polymers; and
b) optionally blending the solid dispersion formulation with one or more of a diluent, disintegrant, salt, lubricant, and glidant, and
c) optionally granulating to form a blended material; and
2) blending Compound I, the blended material, and optionally one or more of a diluent, disintegrant, salt, lubricant, and glidant, and optionally granulating to provide a blended composition, where Compound I is (2R)-isopropyl 2-(((((2R,3R,4R,5R)-4-chloro-5-(2,4-dioxo-3,4-dihydropyrimidin-1(2H)-yl)-3-hydroxy-4-methyltetrahydrofuran-2-yl)methoxy) (phenoxy)phosphoryl)amino)propanoate: 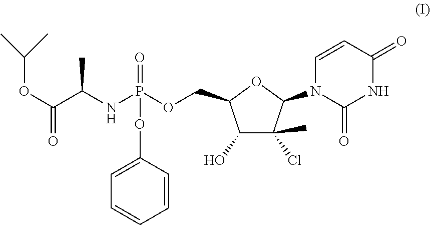 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]
or a pharmaceutically acceptable salt thereof; and
3) compressing the blend of step 2) into a tablet or filling into a capsule.

15. The process according to claim 14, further comprising film-coating the tablet.
